(19) United States
(12) Patent Application Publication
(10) Pub. No.: US 2025/0329459 A1
(43) Pub. Date: Oct. 23, 2025

(54) METHOD FOR PROVIDING PROGNOSTIC INFORMATION ON HEART FAILURE AND DEVICE FOR PROVIDING THE SAME

(71) Applicant: ONTACT HEALTH CO., LTD., Seoul (KR)

(72) Inventors: Seung Ah Lee, Seoul (KR); Jae Ik Jeon, Uijeongbu-si (KR)

(73) Assignee: ONTACT HEALTH CO., LTD., Seoul (KR)

(21) Appl. No.: 19/186,711

(22) Filed: Apr. 23, 2025

(30) Foreign Application Priority Data
Apr. 23, 2024 (KR) 10-2024-0054014
Apr. 23, 2025 (KR) 10-2025-0053085

(51) Int. Cl. G16H 50/20; A61B 8/00; A61B 8/08; G16H 10/60; G16H 50/30

(52) U.S. Cl. CPC G16H 50/20; A61B 8/0883; A61B 8/461; G16H 10/60; G16H 50/30

(57) ABSTRACT

The present disclosure provides a method for providing prognostic information on heart failure implemented by a processor and a device for providing prognostic information on heart failure using the same, and the method includes receiving an echocardiographic video image of an individual suffering from heart failure and determining data on the prognosis of heart failure based on the received echocardiographic video image, using a prediction model trained to output data on the prognosis of the heart failure by taking the echocardiographic video image as a single input without any other input.

### CROSS-REFERENCE TO RELATED APPLICATIONS

This application claims the priority of Korean Patent Application No. 10-2025-0053085 filed on Apr. 23, 2025 and No.10-2024-0054014 filed on Apr. 23, 2024 in the Korean Intellectual Property Office, the disclosure of which is incorporated herein by reference.

### BACKGROUND

### Field

The present disclosure relates to a method for providing prognostic information on heart failure and a device for providing prognostic information on heart failure using the same.

### Description of the Related Art

Heart failure (HF) is a complex clinical syndrome caused by structural or functional disorders in the contractile or diastolic function of the ventricle, and is mainly accompanied by symptoms such as dyspnea, fatigue, and lower extremity edema, as well as abnormalities in various physiological indicators.

At this time, the prevalence and incidence of the heart failure are continuously increasing along with the increase in risk factors such as the aging population, hypertension, diabetes, and ischemic heart disease, and accordingly, the consumption of medical resources, hospitalization rate, readmission rate, and mortality rate are also maintaining high levels.

In particular, for patients with moderate to severe heart failure, the mortality rate within one year is reported to be over 30%, which is a worse prognosis than that of some high-risk malignant tumors.

This type of heart failure is not limited to a functional problem of the heart, but is often accompanied by multi-organ dysfunction such as renal dysfunction, systemic inflammation, sarcopenia, and malnutrition, which has a significant impact on the patient's quality of life and survival rate.

Accordingly, there is an increasing need for technology that may accurately predict the risk of heart failure patients and quantitatively evaluate their prognosis, and there is a continuous demand for the development of a new information provision system that may interpret the patient's condition and provide prognosis based on more quantitative and intuitive information.

The background technology of the present disclosure has been written to facilitate understanding of the present disclosure. It should not be understood that the matters described in the background technology of the disclosure are recognized as prior art.

### SUMMARY

The inventors of the present disclosure noted that heart failure is a clinical syndrome resulting from structural or functional abnormalities of the heart.

Accordingly, the inventors of the present disclosure have been recognized that in order to quantitatively predict the prognosis of patients with heart failure and establish treatment strategies, in addition to existing static clinical indicators or biochemical markers, indicators reflecting the dynamic changes and overall characteristics of cardiac function over time are needed.

Meanwhile, most of the conventional artificial neural network-based prognostic prediction models only use standardized clinical variables such as blood nitrogen concentration (BUN), sodium concentration, systolic blood pressure (SBP), heart rate, and concomitant diseases, or in some cases, left ventricular ejection fraction (LV-EF) and cardiac function parameters defined by specialists as auxiliary parameters.

However, prognostic prediction systems based on these models may have limitations in accurately predicting prognosis in actual clinical situations because they may not comprehensively quantify the dynamic characteristics of complex cardiac motion.

To overcome these limitations, the inventors of the present disclosure have focused on automatically extracting visual and temporal information embedded in an echocardiographic video image using a deep learning-based artificial neural network algorithm, and predicting the prognosis of heart failure patients based on the extracted information.

In particular, the inventors of the present disclosure noted that, in addition to traditional parameters such as ejection fraction and cardiac volume, a prediction model may train new imaging features not directly defined by experts to predict the prognosis.

Furthermore, the inventors of the present disclosure have noted that the more human opinions are involved in the diagnostic performance of a prediction model, such as hand-designed features designed by most prognostic experts, such as MAGGIC, EFFECT, and ESCAPE, the more the prediction performance is limited.

Accordingly, the inventors of the present disclosure have attempted to build a prediction system with higher reliability and generalization performance by using the echocardiographic video image itself as input without an expert's interpretation-based indicator.

As a result, the inventors of the present disclosure have developed an information provision system that may more reliably predict the prognosis associated with mortality and readmission rates of heart failure patients using only the echocardiographic video image.

Accordingly, the inventors of the present disclosure have expected that by providing a new information provision system, it could contribute to improving the survival rate of patients by providing more practical and intuitive predictive information to clinicians in the diagnosis and treatment decision-making process.

Therefore, an object of the present disclosure is to provide an information providing method configured to receive an echocardiographic video image and predict the prognosis of heart failure for an individual using a prediction model, and a device using the same.

Objects of the present disclosure are not limited to the objects mentioned above, and other objects not mentioned will be clearly understood by those skilled in the art from the description below.

In order to achieve the aforementioned objects, a method for providing prognostic information on heart failure according to one embodiment of the present disclosure is provided.

The information providing method is implemented by a processor and includes receiving an echocardiographic video image of an individual suffering from heart failure, and determining data on the prognosis of heart failure based on the received echocardiographic video image, using a prediction model trained to output data on the prognosis of the heart failure by taking the echocardiographic video image as a single input without any other input.

According to an aspect of the present disclosure, the echocardiographic video image may be a cardiac ultrasound video including a plurality of frames, and the prediction model may include a 3D encoder configured to extract features for each of a plurality of received frames, and a transformer configured to derive a temporal relationship between the plurality of frames so that an integrated time series feature is generated.

According to another aspect of the present disclosure, the prediction model may further include a spatial attention pooling configured to generate an integrated spatial feature by considering adjacent frame features for one selected frame among the plurality of frames.

According to another aspect of the present disclosure, the data on the prognosis of the heart failure may include at least one of a survival probability within a predetermined period, a cumulative survival curve for the survival probability, a hazard ratio, and a mortality risk score.

According to another aspect of the present disclosure, the data on the prognosis of the heart failure may be provided in the form of a graphical user interface (GUI) in which a mortality risk at each time point is visually expressed.

According to another aspect of the present disclosure, sex and age for the individual may be additionally received, and the data on the prognosis of the heart failure may be determined based on the received sex, age, and echocardiographic video image using the prediction model.

According to another aspect of the present disclosure, a first feature for the received sex and age may be extracted, a second feature for the received echocardiographic video image may be extracted, the first feature and the second feature may be integrated, and the data for the prognosis of the heart failure may be determined based on the integrated feature.

According to another aspect of the present disclosure, the prediction model may be constructed as a model that binary-classifies whether the individual survives for each of a plurality of time intervals and is trained to generate a survival probability function by accumulating a binary classification result according to whether the individual survives for each time interval.

In order to achieve the aforementioned objects, a device for providing prognostic information on heart failure according to another embodiment of the present disclosure is provided.

The information providing device may include a communication unit configured to receive an echocardiographic video image of an individual suffering from heart failure, and a processor functionally connected to the communication unit, in which the processor determines data on the prognosis of heart failure based on the received echocardiographic video image using a prediction model trained to output data on the prognosis of heart failure by taking the echocardiographic video image as a single input without any other input.

According to an aspect of the present disclosure, the communication unit may be configured to additionally receive sex and age of the individual, and the processor may determine the data on the prognosis of the heart failure based on the received sex, age, and echocardiographic video image using the prediction model.

According to another aspect of the present disclosure, the processor may be further configured to extract a first feature for the received sex and age using the prediction model, extract a second feature for the received echocardiographic video image, integrates the first feature and the second feature, and determine the data on the prognosis of the heart failure based on the integrated feature.

Specific details of other embodiments are included in the detailed description and drawings.

The present disclosure can provide a deep learning-based information provision system that automatically extracts visual and temporal information included in the echocardiographic video image and predicts the prognosis of a heart failure patient based on the extracted information.

Accordingly, the present disclosure can overcome the limitations of existing clinical indicator-based prediction models, for example, the limitations of existing systems that could not sufficiently reflect the complex dynamics of cardiac function by utilizing only fragmentary indicators such as blood nitrogen concentration, sodium concentration, systolic blood pressure, and left ventricular ejection fraction.

In particular, the present disclosure enables more precise prognosis prediction by utilizing image-based features based on the prediction model using only the echocardiographic video images, without relying solely on existing indicators defined by medical experts.

In this regard, the present disclosure provides a prediction model that extracts features associated with prognosis from the image itself and outputs information on prognosis without the interpretation of a clinician, thereby providing highly reliable information.

That is, the present disclosure can provide consistent prediction results that do not depend on the subjective judgment of a clinician by analyzing temporal changes and functional patterns of cardiac movement, which were difficult to quantify, by a prediction model.

Therefore, the information providing system of the present disclosure can provide prognostic information on heart failure patients, such as mortality and readmission, using only the echocardiographic video images, thereby simplifying examination time and analysis procedures compared to conventional systems.

Accordingly, the present disclosure may have high applicability in medical fields.

Furthermore, the present disclosure can contribute to improving the accuracy of prognosis prediction for a heart failure patient, supporting diagnosis and treatment decision-making, and improving the survival rate and quality of life of patients.

The effects according to the present disclosure are not limited to those exemplified above, and further diverse effects are included in the present specification.

The effects of the present disclosure are not limited to the aforementioned effects, and other effects, which are not mentioned above, will be apparently understood to a person having ordinary skill in the art from the following description.

The objects to be achieved by the present disclosure, the means for achieving the objects, and the effects of the present disclosure described above do not specify essential features of the claims, and, thus, the scope of the claims is not limited to the disclosure of the present disclosure.

### BRIEF DESCRIPTION OF DRAWINGS

The above and other aspects, features and other advantages of the present disclosure will be more clearly understood from the following detailed description taken in conjunction with the accompanying drawings, in which:

FIG. 1 illustrates a system for providing prognostic information on heart failure using a device for providing prognostic information on heart failure according to one embodiment of the present disclosure;

FIG. 2A is a block diagram illustrating the configuration of a medical staff device according to one embodiment of the present disclosure;

FIG. 2B is a block diagram illustrating the configuration of an information providing server according to one embodiment of the present disclosure;

FIGS. 3A to 3D and FIGS. 4A to 4C illustrate procedures of a method for providing prognostic information on heart failure according to various embodiments of the present disclosure; and

FIGS. 5A to 5F illustrate evaluation results of a prediction model used in an information providing method according to various embodiments of the present disclosure.

### DETAILED DESCRIPTION OF THE EMBODIMENT

Hereinafter, the exemplary embodiment of the present disclosure will be described with reference to the accompanying drawings and exemplary embodiments as follows. Scales of components illustrated in the accompanying drawings are different from the real scales for the purpose of description, so that the scales are not limited to those illustrated in the drawings.

The advantages of the disclosure and the method for achieving them will become apparent by referring to the embodiments described in detail below together with the accompanying drawings. However, the present disclosure is not limited to the embodiments disclosed below, but may be implemented in various different forms, and these embodiments are provided only to make the disclosure of the present disclosure complete and to fully inform those skilled in the art of the scope of the disclosure.

The shapes, sizes, ratios, angles, numbers, or the like disclosed in the drawings for explaining embodiments of the present disclosure are exemplary, and therefore the present disclosure is not limited to the matters illustrated. In addition, when describing the present disclosure, if it is determined that a detailed description of a related known technology may unnecessarily obscure the gist of the present disclosure, the detailed description will be omitted. When the terms “include”, “have”, “consist of”, and the like are used in this specification, other parts may be added unless “only” is used. When a component is expressed in singular, it includes a case where the plural is included unless there is a specifically explicit description.

When interpreting components, it is interpreted as including the error range even when there is no separate explicit description.

The individual features of the various embodiments of the present disclosure may be partially or wholly combined or combined with each other, and as may be fully understood by those skilled in the art, various technical connections and operations are possible, and each embodiment may be implemented independently of each other or may be implemented together in a related relationship.

For clarity in the interpretation of this specification, the terms used in this specification are defined below.

The term “individual” as used herein refers to any subject for whom a prognosis of heart failure is sought from echocardiographic video images, preferably an individual suffering from heart failure. Meanwhile, the individual disclosed in this specification may be any mammal other than a human, but is not limited thereto.

As used in the present disclosure, the term “echocardiographic video image” may be a single frame still cut image or a video including multiple frames, as a prognosis for heart failure in an individual.

At this time, the echocardiographic video image may be a 2D or 3D image.

For example, in one embodiment of the present disclosure, the echocardiographic video image may be in the form of a video including a plurality of frames and may include temporal change information of the heart including contraction and relaxation processes, but is not limited thereto.

The term “prediction model” as used in the present specification may mean a deep learning-based artificial neural network model that takes echocardiographic video images and/or individual information as input and outputs data on the prognosis of heart failure.

In one embodiment of the present disclosure, the prediction model may include a 3D encoder for extracting an embedding vector from each of a plurality of frames included in the echocardiographic video image in a video format, a spatial attention pooling for generating features by reflecting information of temporally adjacent frames together, and a transformer for generating integrated features by reflecting temporal correlations between time-series features.

Here, the “3D encoder” may mean a deep learning-based encoder that extracts a feature vector of each frame from the echocardiographic video image including multiple frames.

For example, in one embodiment of the present disclosure, the 3D encoder is based on a 3D CNN structure and may generate an embedding that simultaneously reflects time and space information. However, the present disclosure is not limited thereto.

The term “spatial attention pooling” as used in the present specification may be a unit configured to generate an integrated spatial feature by considering adjacent frame features for one selected frame among a plurality of frames.

The term “transformer” as used in the present specification may mean a self-attention-based neural network unit that analyzes and integrates temporal relationships between the plurality of frames or feature vectors.

In one embodiment of the present disclosure, the transformer may utilize frame-level or video-level information to derive a temporal relationship for each of the plurality of frames to generate integrated time-series features, but is not limited thereto.

Meanwhile, the structure of the prediction model is not limited to the aforementioned configuration, and may be constructed in various forms as long as it takes a video-format echocardiographic video image as a single input, extracts motion-based features, and outputs data on the prognosis of heart failure.

For example, the prediction model may include a 2-D encoder that extracts two-dimensional features for each frame, and may include a recurrent neural network series processing unit or a time series processing unit that processes the extracted frame-by-frame features by organizing them into a time series.

Furthermore, the prediction model may include a convolutional LSTM structure configured to intensively train features only for selected regions of interest within the echocardiographic image, or a structure that nonlinearly trains relationships between frames.

Furthermore, the prediction model may include a structure that extracts features using a pre-trained Vision Transformer (ViT) or a non-linear mechanism.

Meanwhile, in various embodiments of the present disclosure, the prediction model may additionally receive individual information, such as the sex and age of the individual, and at this time, extract a first feature from the received individual information, extract a second feature from an echocardiographic video image, and then perform prognosis prediction by integrating the two features.

In more various embodiments of the present disclosure, the prediction model may be trained in a manner of binary classifying whether the individual survives for each of a plurality of time intervals, and may be trained in a manner of accumulating the classification results for each time interval to construct a survival probability function.

For example, in one embodiment of the present disclosure, the prediction model may be a model trained based on a discrete-time binary classification loss function for survival, but is not limited thereto, and may be a model trained based on various other methods, such as a survival loss function based on partial log likelihood (negative log partial likelihood).

In more various embodiments of the present disclosure, the prediction model may be configured to extract motion features reflecting temporal context by considering not only a single frame independently, but also frames of preceding and following time intervals, that is, context information.

For example, the prediction model may be modeled to receive information from adjacent previous and subsequent frames centered around the current frame, and generate spatiotemporal features that more precisely represent the state of cardiac function at that point in time.

Due to these features, the prediction model may reflect the changing patterns of cardiac structure, so the accuracy of prognosis prediction may be superior to models that do not.

Meanwhile, the prediction model may output data on more various heart failure prognosis.

The term “data on the prognosis of heart failure” as used in this specification may mean prediction results including survival-related information output by a prediction model, such as the long-term/short-term survival probability of the individual, mortality risk, or probability of death over time.

For example, in one embodiment of the present disclosure, the data on the prognosis of heart failure may include, but is not limited to, prediction results in the form of a survival probability within a preset period, a cumulative survival curve over time, a hazard ratio, a mortality risk score, or a graphical user interface visually representing the same. However, the present disclosure is not limited to this.

The term “survival probability function” as used in this specification may mean a cumulative survival function that expresses the probability of an individual surviving until a specific point in time as a function of time.

In further various embodiments of the present disclosure, the data on the prognosis of heart failure may be in the form of a graphical user interface (GUI) in which the mortality risk at each time point is visually represented.

Accordingly, a user may intuitively understand the survival probability over time, high-

risk areas, or prognostic changes after a specific treatment point, and may make clinical judgments and establish treatment strategies based on the provided prognostic information.

At this time, the graphical user interface may be implemented in the form of, but is not limited to, a cumulative survival curve, a time-point risk score bar graph, or a heat map that visually distinguishes risk by color interval.

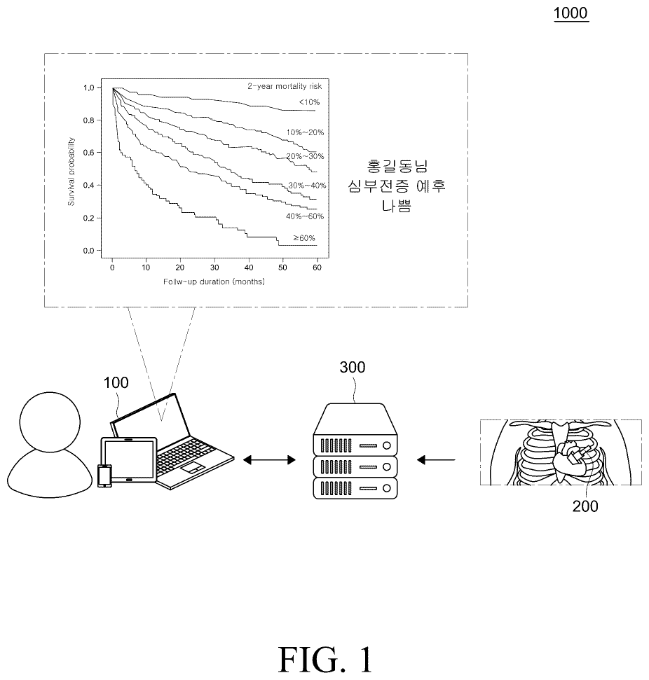

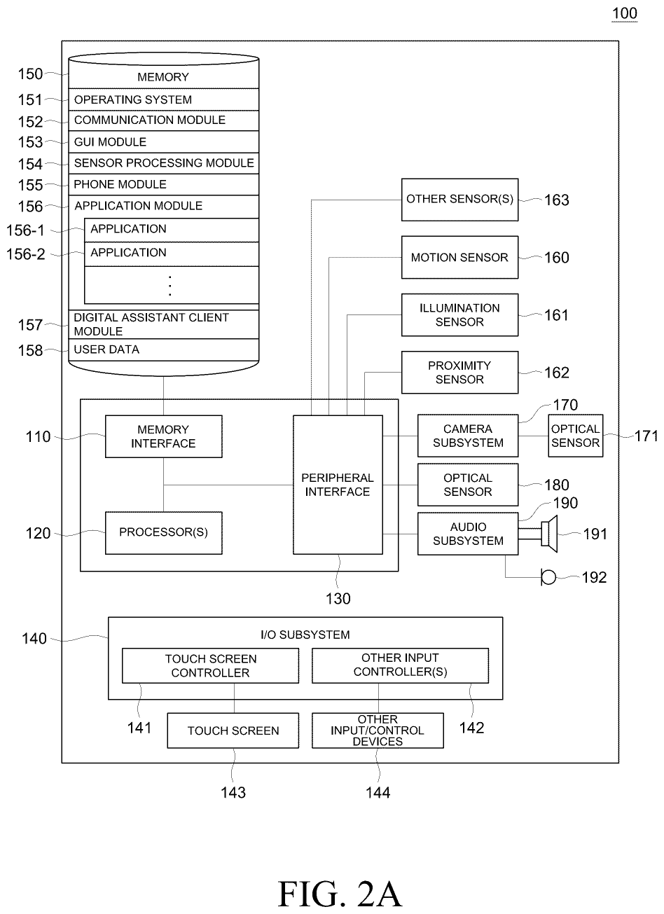

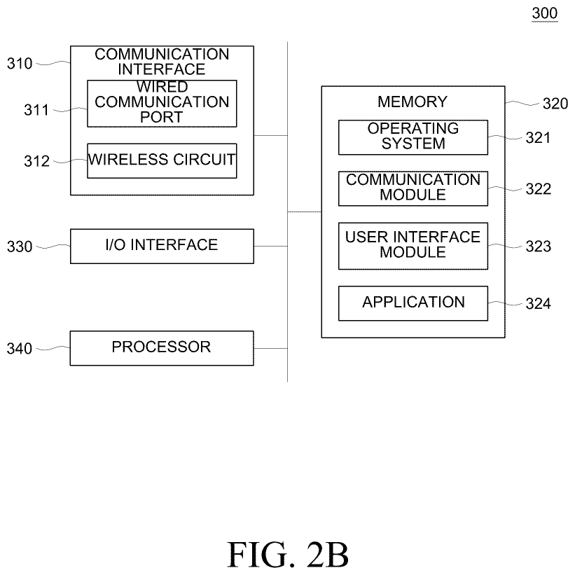

Hereinafter, with reference to FIGS. 1, 2A and 2B, a system for providing prognostic information on heart failure using a device for providing prognostic information on heart failure according to one embodiment of the present disclosure and a device for providing prognostic information on heart failure will be described.

FIG. 1 illustrates a system for providing prognostic information on heart failure using a device for providing prognostic information on heart failure according to one embodiment of the present disclosure. FIG. 2A illustrates an exemplary configuration of a medical staff device for receiving guidelines on the prognosis of heart failure according to one embodiment of the present disclosure. FIG. 2B illustrates an exemplary configuration of a server for providing prognostic information on heart failure according to one embodiment of the present disclosure.

First, referring to FIG. 1, an information providing system 1000 may be a system configured to calculate heart failure prognosis data based on an echocardiographic video image of an individual and provide survival probability or risk information related thereto.

At this time, the information providing system 1000 may include an ultrasound imaging diagnosis device 200 that captures and provides an echocardiographic video image, an information providing server 300 that outputs prognostic data through a prediction model, and a medical staff device 100 that visually receives prognostic information.

In various embodiments of the present disclosure, the information providing server 300 may be integrally mounted within the ultrasound imaging diagnosis device 200, and in this case, the prognostic data may be displayed in real time on a display unit (not illustrated) of the ultrasound imaging diagnosis device 200.

For example, a survival curve, cumulative risk, or mortality risk score for the individual may be visually presented simultaneously with the imaging.

That is, the user may use the ultrasound imaging diagnostic device 200 to check information related to the prognosis of heart failure at the same time as the diagnosis.

In various embodiments, the medical staff device 100 is an electronic device that provides a user interface for presenting information associated with the measurement parameter, and may include at least one of a smart phone, a tablet PC (personal computer), a laptop, and/or a PC.

Meanwhile, the information providing server 300 may include a general-purpose computer, laptop, and/or data server that calculates data on the prognosis of heart failure based on an echocardiographic video image provided from the ultrasound imaging diagnostic device 200, such as an ultrasound diagnostic device, and performs various operations to generate information based on the calculated data.

In this case, the information providing server 300 may be a device for accessing a web server providing a web page or a mobile web server providing a mobile website, but is not limited thereto.

More specifically, the information providing server 300 receives an echocardiographic video image from an ultrasound imaging diagnostic device 200 and applies a prediction model to the received image to calculate the heart failure prognosis data.

At this time, the information providing server 300 may predict the heart failure prognosis data including at least one of the survival probability, cumulative survival curve, hazard ratio, or mortality risk score based on the echocardiographic video image and, if necessary, sex and age information received using the prediction model.

Furthermore, the information providing server 300 may generate intuitive diagnostic support information, such as changes in survival probability by time point, high-risk intervals, or risk grades, based on predicted prognosis data.

The information providing server 300 may provide the prognostic information and visualized prediction results, or guideline information to the medical staff device 100.

Next, the components of the information providing server 300 of the present disclosure will be specifically described with reference to FIGS. 2A and 2B.

First, referring to FIG. 2A, the medical staff device 100 may include a memory interface 110, one or more processors 120, and a peripheral interface 130. Various components within the medical staff device 100 may be connected by one or more communication buses or signal lines.

The memory interface 110 may be connected to a memory 150 and transmit various data to a processor 120. Here, the memory 150 may include at least one type of storage medium among a flash memory type, a hard disk type, a multimedia card micro type, a card type memory (for example, SD or XD memory, or the like), a RAM, an SRAM, a ROM, an EEPROM, a PROM, a network storage, cloud, and blockchain data.

In various embodiments, the memory 150 may store at least one of an operating system 151, a communication module 152, a graphical user interface module (GUI) 153, a prognostic data processing module 154, a telephone module 155, and an application module 156. Specifically, the operating system 151 may include instructions for processing basic system services and instructions for performing hardware operations. The communication module 152 may communicate with at least one of other devices, computers, and servers. The graphical user interface module (GUI) 153 may process an interface for visually displaying data regarding heart failure prognosis.

The data processing module 154 may internally calculate the heart failure prognosis data received from the server or perform a conversion task for outputting the heart failure prognosis data on the GUI. The phone module 155 may process phone-related functions. The application module 156 may perform various functions of the user application, such as electronic messaging, web browsing, media processing, navigation, imaging, and other processing functions. In addition, the medical staff device 100 may store one or more software applications 156-1 and 156-2 (for example, a heart failure prognosis information providing application) associated with a type of service in the memory 150.

In various embodiments, the memory 150 may store a digital assistant client module 157 (hereinafter, DA client module), and thus store instructions for performing client-side functions of the digital assistant and various user data 158.

Meanwhile, the DA client module 157 may obtain the user's voice input, text input, touch input, and/or gesture input through various user interfaces (for example, I/O subsystem 140) provided in the medical staff device 100.

Additionally, the DA client module 157 may output data in audiovisual and tactile forms. For example, the DA client module 157 may output data consisting of a combination of at least two or more of voice, sound, notification, text message, menu, graphic, video, animation, and vibration. Additionally, the DA client module 157 may communicate with a digital assistant server (not illustrated) using a communication subsystem 180.

In various embodiments, the DA client module 157 may collect additional information about the surroundings of the medical staff device 100 from various sensors, subsystems, and peripheral devices to construct a context associated with the user input. For example, the DA client module 157 may provide context information along with the user input to a digital assistant server to infer the user's intent. Here, the context information that may accompany the user input may include sensor information, such as lighting, ambient noise, ambient temperature, images of the surrounding environment, video, or the like. As another example, the context information may include the physical state of the medical staff device 100 (for example, device orientation, device position, device temperature, power level, speed, acceleration, motion pattern, cellular signal strength, or the like). As another example, the context information may include information related to the software state of the medical staff device 100 (for example, processes running on the medical staff device 100, installed programs, past and current network activity, background services, error logs, resource usage, or the like).

In various embodiments, the memory 150 may include additional or deleted instructions, and further, the medical staff device 100 may include additional configurations other than those illustrated in FIG. 2A, or may exclude some configurations.

The processor 120 may control the overall operation of the medical staff device 100 and execute various commands to implement a user interface that visually provides information related to the prognosis of heart failure by running an application or program stored in the memory 150.

The processor 120 may correspond to a computational unit such as a central processing unit (CPU) or an application processor (AP). In addition, the processor 120 may be implemented in the form of an integrated chip (IC) such as a system on chip (SoC) in which various computational units such as a neural processing unit (NPU) are integrated.

Next, referring to FIG. 2B, the information providing server 300 may include a communication interface 310, a memory 320, an I/O interface 330, and a processor 340, each component of which may communicate with each other through one or more communication buses or signal lines.

The communication interface 310 may be connected to the medical staff device 100 and the ultrasound imaging diagnosis device 200 via a wired/wireless communication network to exchange data. For example, the communication interface 310 may receive the echocardiographic video image from the ultrasound imaging diagnosis device 200, determine measurement parameters or guidelines for obtaining the same therefrom, and transmit the parameters or guidelines to the medical staff device 100.

Meanwhile, the communication interface 310 that enables transmission and reception of such data includes a communication port 311 and a wireless circuit 312, in which the wired communication port 311 may include one or more wired interfaces, for example, Ethernet, Universal Serial Bus (USB), FireWire, or the like. In addition, the wireless circuit 312 may transmit and receive data with an external device via an RF signal or an optical signal. In addition, the wireless communication may use at least one of a plurality of communication standards, protocols, and technologies, for example, GSM, EDGE, CDMA, TDMA, Bluetooth, Wi-Fi, VoIP, Wi-MAX, or any other suitable communication protocol.

The memory 320 may store various data used in the information providing server 300. For example, the memory 320 may store the heart failure prognosis or the prediction model trained to segment anatomical structures within the heart failure prognosis.

In various embodiments, the memory 320 may include a volatile or nonvolatile storage medium capable of storing various data, commands, and information. For example, the memory 320 may include at least one type of storage medium among a flash memory type, a hard disk type, a multimedia card micro type, a card type memory (for example, an SD or XD memory, or the like), a RAM, an SRAM, a ROM, an EEPROM, a PROM, a network storage, a cloud, and blockchain data.

In various embodiments, the memory 320 may store a configuration of at least one of an operating system 321, a communication module 322, a user interface module 323, and one or more applications 324.

The operating system 321 (for example, an embedded operating system such as LINUX, UNIX, MAC OS, WINDOWS, VxWorks, or the like) may include various software components and drivers to control and manage general system operations (for example, memory management, storage device control, power management, or the like) and may support communication between various hardware, firmware, and software components.

The communication module 323 may support communication with other devices through the communication interface 310. The communication module 320 may include various software components for processing data received by the wired communication port 311 or wireless circuit 312 of the communication interface 310.

The user interface module 323 may receive a user's request or input from a keyboard, touch screen, microphone, or the like through an I/O interface 330 and provide a user interface on the display.

The application 324 may include a program or module configured to be executed by one or more processors 330. Here, the application for providing information associated with measurement parameters may be implemented on a server farm.

The I/O interface 330 may connect an input/output device (not illustrated) of the information providing server 300, such as at least one of a display, a keyboard, a touch screen, and a microphone, to the user interface module 323. The I/O interface 330 may receive user input (for example, voice input, keyboard input, touch input, or the like) together with the user interface module 323 and process a command according to the received input.

The processor 340 may be connected to the communication interface 310, the memory 320, and the I/O interface 330 to control the overall operation of the information providing server 300, and perform various commands for providing information through an application or program stored in the memory 320.

The processor 340 may correspond to a computational device such as a central processing unit (CPU) or an application processor (AP). In addition, the processor 340 may be implemented in the form of an integrated chip (IC) such as a system on chip (SoC) in which various computational devices are integrated. Alternatively, the processor 340 may include a module for calculating an artificial neural network model such as a neural processing unit (NPU).

In various embodiments, the processor 340 may be configured to predict anatomical structures within the heart failure prognosis using prediction models and automatically determine and provide measurements. Optionally, the processor 340 may be configured to provide information about obtainable measurements from the heart failure prognosis and information about possible modes of measurement.

Next, referring to FIG. 2B, the information providing server 300 may include the communication interface 310, the memory 320, the I/O interface 330, and the processor 340, each component of which may communicate with each other through one or more communication buses or signal lines.

The communication interface 310 may be connected to the medical staff device 100 and the ultrasound imaging diagnosis device 200 via a wired/wireless communication network to exchange data. For example, the communication interface 310 may receive the echocardiographic video image from the ultrasound imaging diagnosis device 200, calculate the data on the prognosis of heart failure therefrom, and transmit the prognosis result and corresponding information to the medical staff device 100.

Meanwhile, the communication interface 310 that enables transmission and reception of such data includes the communication port 311 and the wireless circuit 312, in which the wired communication port 311 may include one or more wired interfaces, for example, Ethernet, Universal Serial Bus (USB), FireWire, or the like. In addition, the wireless circuit 312 may transmit and receive data with an external device via an RF signal or an optical signal. In addition, the wireless communication may use at least one of a plurality of communication standards, protocols, and technologies, for example, GSM, EDGE, CDMA, TDMA, Bluetooth, Wi-Fi, VoIP, Wi-MAX, or any other suitable communication protocol.

The memory 320 may store various data used in the information providing server 300. For example, the memory 320 may store the prediction model trained to output echocardiographic video images, predicted heart failure prognosis, or prognosis data.

In various embodiments, the memory 320 may include a volatile or nonvolatile storage medium capable of storing various data, commands, and information. For example, the memory 320 may include at least one type of storage medium among a flash memory type, a hard disk type, a multimedia card micro type, a card type memory (for example, an SD or XD memory, or the like), a RAM, an SRAM, a ROM, an EEPROM, a PROM, a network storage, a cloud, and blockchain data.

In various embodiments, the memory 320 may store a configuration of at least one of the operating system 321, the communication module 322, the user interface module 323, and one or more applications 324.

The operating system 321 (for example, an embedded operating system such as LINUX, UNIX, MAC OS, WINDOWS, VxWorks, or the like) may include various software components and drivers to control and manage general system operations (for example, memory management, storage device control, power management, or the like) and may support communication between various hardware, firmware, and software components.

The communication module 322 may support communication with other devices through the communication interface 310. The communication module 322 may include various software components for processing data received by the wired communication port 311 or wireless circuit 312 of the communication interface 310.

The user interface module 323 may receive a user's request or input from a keyboard, touch screen, microphone, or the like through the I/O interface 330 and provide the user interface on the display.

The application 324 may include a program or module configured to be executed by one or more processors 340.

Here, the application for analyzing and providing the heart failure prognosis data may be implemented on a server farm.

The I/O interface 330 may connect an input/output device (not illustrated) of the information providing server 300, such as at least one of a display, a keyboard, a touch screen, and a microphone, to the user interface module 323. The I/O interface 330 may receive user input (for example, voice input, keyboard input, touch input, or the like) together with the user interface module 323 and process a command according to the received input.

The processor 340 may be connected to the communication interface 310, the memory 320, and the I/O interface 330 to control the overall operation of the information providing server 300, and may perform various commands for providing information through an application or program stored in the memory 320.

The processor 340 may correspond to a computational device such as a central processing unit (CPU) or an application processor (AP). In addition, the processor 340 may be implemented in the form of an integrated chip (IC) such as a system on chip (SoC) in which various computational devices are integrated. Alternatively, the processor 340 may include a module for calculating an artificial neural network model such as a neural processing unit (NPU).

In various embodiments, the processor 340 may be configured to predict the heart failure prognosis from echocardiographic video images using the prediction models, and to calculate and provide the survival probability, the cumulative survival curve, or the mortality risk score based on the predicted prognosis data.

Optionally, the processor 340 may be configured to generate customized clinical judgment support information or visual user interface output based on the heart failure prognosis.

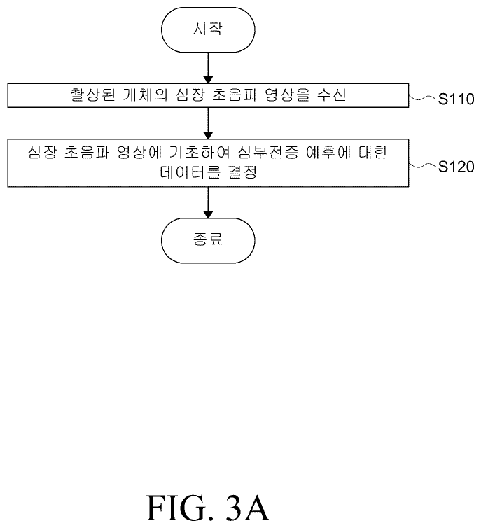

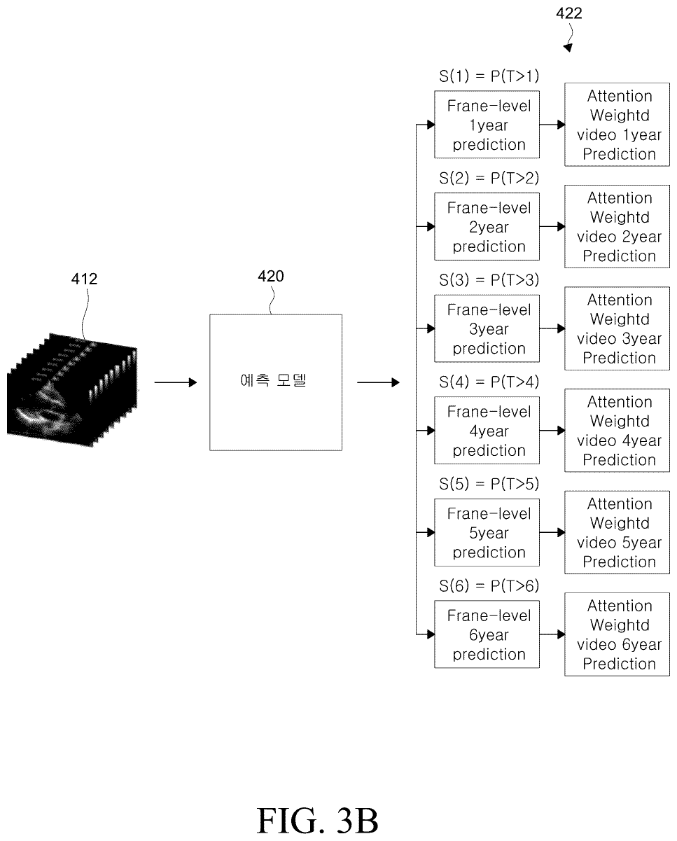

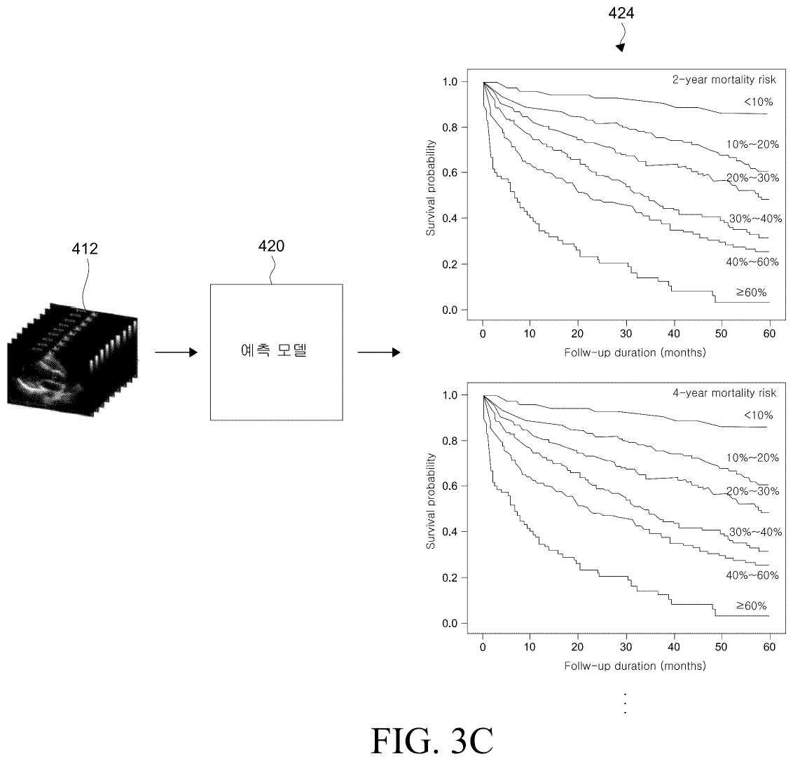

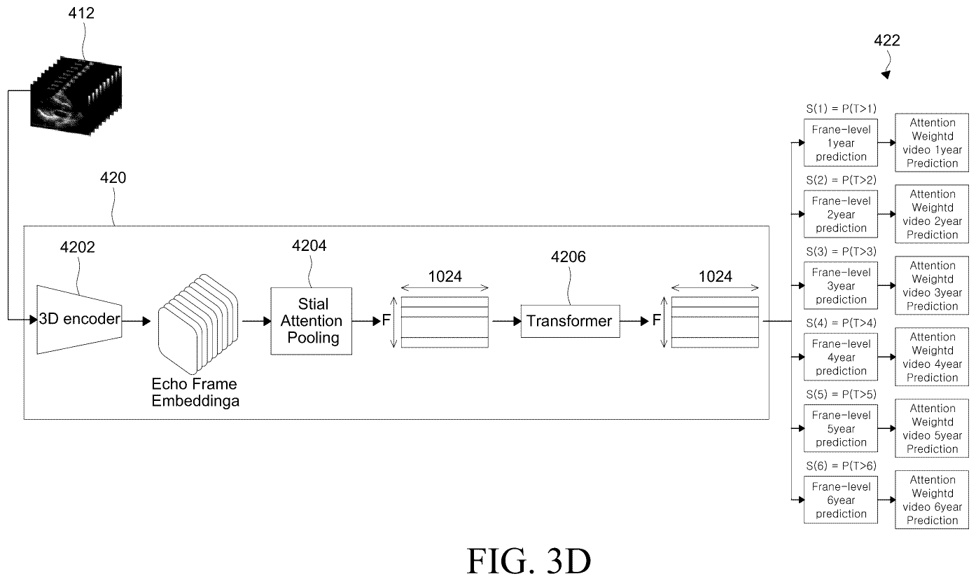

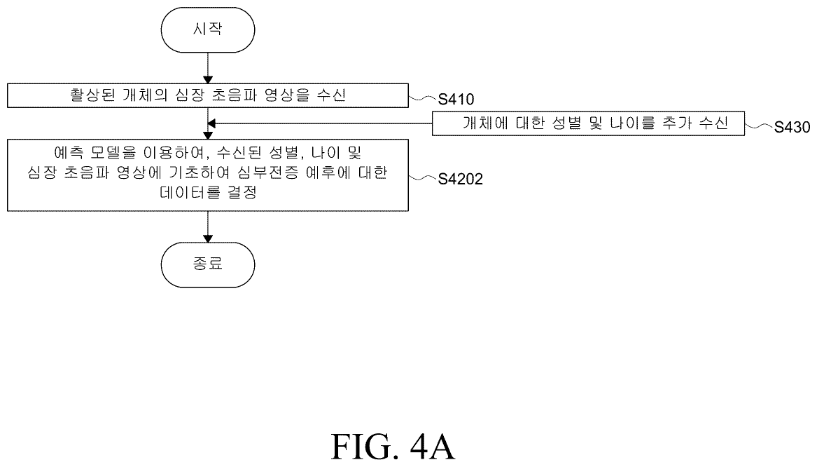

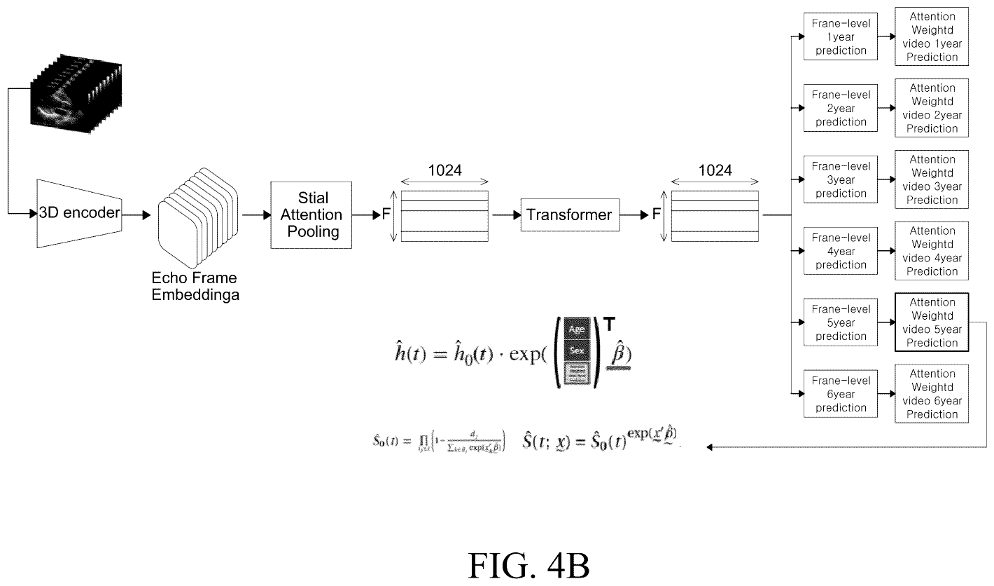

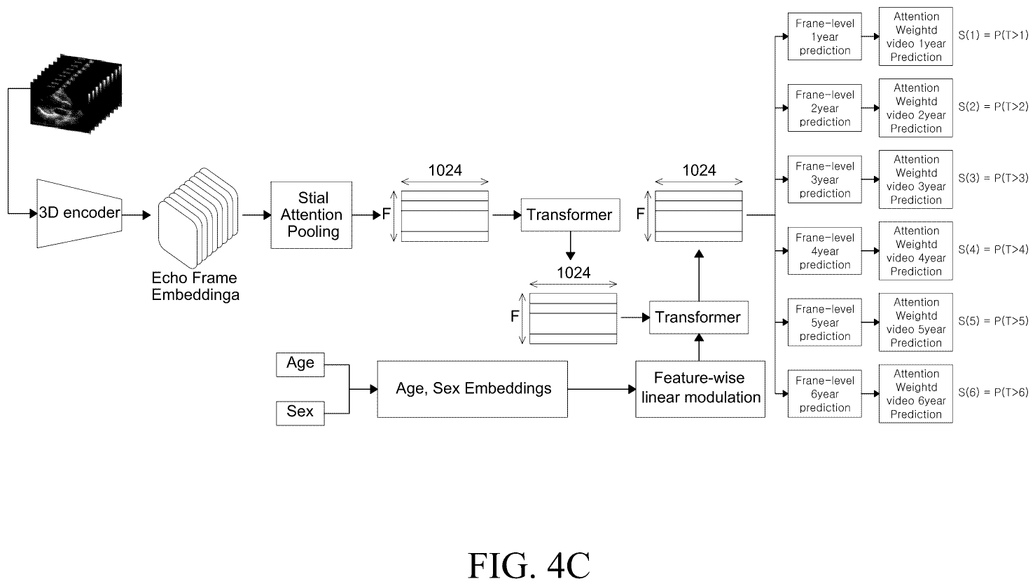

Hereinafter, with reference to FIGS. 3A to 3D and FIGS. 4A to 4C, a method for providing the prognostic information on heart failure and a configuration of a model according to one embodiment of the present disclosure will be specifically described.

FIGS. 3A to 3D and FIGS. 4A to 4C illustrate procedures of the method for providing the prognostic information on heart failure according to various embodiments of the present disclosure.

First, referring to FIG. 3A, the information provision procedure according to one embodiment of the present disclosure is as follows. First, an echocardiographic video image of an individual is received (S310). Then, the data on the prognosis of heart failure is determined by the prediction model (S320).

According to an aspect of the present disclosure, the echocardiographic video image may be the cardiac ultrasound video including a plurality of frames.

According to an aspect of the present disclosure, in Step (S320) in which the data on the prognosis of heart failure is determined, the data on the prognosis of heart failure may be at least one of, but is not limited to, a survival probability within a predetermined period, a cumulative survival curve for the survival probability, a hazard ratio, and a mortality risk score.

Furthermore, the data on the prognosis of heart failure may have the form of a graphical user interface (GUI) that visually displays the mortality risk at each time point.

For example, referring to FIG. 3B, in Step S310 in which an echocardiographic video image is received, a cardiac ultrasound video 412 for the individual is received and input into a prediction model 420.

Next, at Step S320 in which the data on the prognosis of heart failure is determined, the prediction model 420 extracts visual and temporal features of heart function on a frame-by-frame basis, and determines and outputs a survival function for multiple survival periods based on the extracted features.

These survival probabilities are predicted frame-by-frame and video-by-video for survival probabilities of 1 year, 2 years, 3 years, 4 years, 5 years, and 6 years or more, and the cumulative risk 422 together with the attention weighted video-level prediction for each time point may be determined as data for the prognosis of heart failure.

The determined heart failure prognosis data may be displayed on the medical staff device in the form of a graphical user interface (GUI) that intuitively visualizes the survival probability within a specific time range, and may be used as quantitative basis information for clinical decision-making or patient counseling.

More specifically, referring to FIG. 3C, when the cardiac ultrasound video 412 is input into the prediction model 420, the prediction model may calculate the survival probabilities for various time intervals and generate and output a survival function of the cumulative survival curve based on the calculated values.

The cumulative survival curve 424 in GUI form is provided by dividing the survival curve into sections according to the level of the death risk within a specific period, such as 2 years or 4 years, and the user may intuitively check the survival pattern according to the death risk group to which the individual belongs.

At this time, in more various embodiments, since each survival curve may be provided in the form of a color or line according to the risk percentage range, the survival curve may be as a visual judgment indicator for establishing a customized treatment strategy or monitoring the patient's condition based on predicted prognosis data.

Meanwhile, in more various embodiments of the present disclosure, the prediction model may include a 3D encoder configured to extract features for each of a plurality of frames of the received 3D echocardiographic video image, and a transformer configured to derive a temporal relationship for each of the plurality of frames to generate an integrated time series feature.

Furthermore, the prediction model may further include a spatial attention pooling configured to generate the integrated spatial feature by considering adjacent frame features for a selected frame among the plurality of frames.

For example, referring to FIG. 3D, when the echocardiographic video image 412 including a plurality of frames in a video format is input to the prediction model 420, features for each frame are first extracted by the 3D encoder 4202.

At this time, each frame generates an embedding vector that reflects the motion characteristics of the cardiac movement, that is the movement over time.

The generated frame-by-frame embedding vector is processed through a spatial attention pooling 4204 to generate an integrated spatial feature by considering not only spatial information for one frame but also the spatiotemporal context of surrounding frames.

Thereafter, the spatial features are transferred to a transformer 4206 to infer the temporal relationship between the frames and the global time series pattern, which may contribute to precise prognosis prediction that reflects the dynamic flow of cardiac function that changes over time rather than a single frame.

Next, the embedding output from the transformer part 4206 moves to the output layer for predicting the prognosis for various survival periods. That is, it is possible to predict the prognosis of heart failure based on time series features with embedded time information through attention-weighted image prediction.

However, the present disclosure is not limited thereto, and the prediction model may have a more various structure to extract temporal features from echocardiographic video images and output prognostic data based on the temporal features.

For example, the prediction model may be constructed based on a two-dimensional or three-dimensional convolutional neural network (CNN), a recurrent neural network (RNN), a transformer, or a combination thereof, and may effectively train patterns of changes in cardiac function over time and perform prognosis prediction considering interactions between frames.

Meanwhile, referring to FIG. 4A, in more various embodiments of the present disclosure, individual data on the sex and age of the individual may be additionally received (S430), and the data on the prognosis of heart failure may be determined based on the received sex, age, and echocardiographic video image using the prediction model (S4202).

More specifically, in Step (S4202) in which the data for the prognosis of heart failure is determined, first features for the received sex and age are extracted using the prediction model, second features for the received echocardiographic video image are extracted, the first features and the second features are integrated, and the data for the prognosis of heart failure may be determined based on the integrated features.

That is, by combining the stereotyped individual information received from the individual and the temporal features automatically extracted from the cardiac ultrasound video images, more precise and personalized heart failure prognosis prediction may be possible.

This method may also improve prediction accuracy and clinical usability by integrating and reflecting various clinical factors compared to existing single-input-based prediction models.

Meanwhile, referring to FIG. 4B, the prediction model may be modeled by incorporating additional variables (sex and age) into the survival prediction results based on time series embedding to integrate the features of sex and age along with the imaging features of cardiac ultrasound.

More specifically, visual and temporal features extracted from the echocardiographic video image are predicted as survival probabilities for each time interval through a transformer, and these prediction results may be combined with additional variables through the statistical structure of the survival model family.

At this time ĥ(t) which is a baseline hazard function over time may be derived from the image-based prediction model, and Ŝ(t; {tilde under (x)}) may be corrected by reflecting the regression coefficient β based on the age and the additional variable of age of the individual as illustrated in the following Mathematical Expression 1.

$$\hat{S} ( t ; {x}_{\sim} ) = {{\hat{S}}_{0} ( t )}^{exp ( {{x}_{\sim}}^{′} {\hat{β}}_{\sim} )} \qquad [ Mathematical Expression 1 ]$$

However, the modeling method of the prediction model is not limited to this.

Referring further to FIG. 4C, the prediction model may also be modeled by linking feature embeddings for individual information, such as sex and age, to the time series embedding output from the transformer.

More specifically, the received sex and age information is converted into an embedding

vector through an encoding process, and the embedding values are integrated into the image-based time series features through a feature-wise linear modulation module.

This structure may quantitatively reflect prognostic patterns that may change depending on sex and age, along with features automatically extracted from images, and may provide a flexible structure that may reflect additional variables for individuals in the deep learning-based model itself.

### Evaluation: Evaluation of Prediction Model

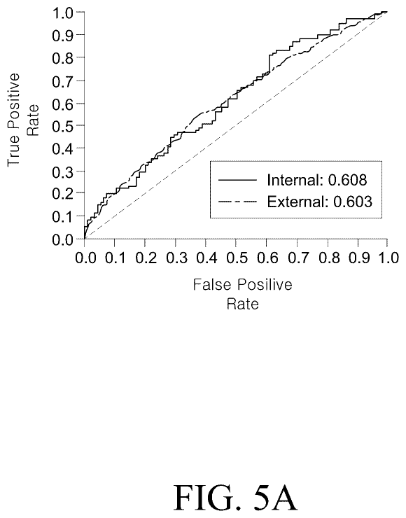

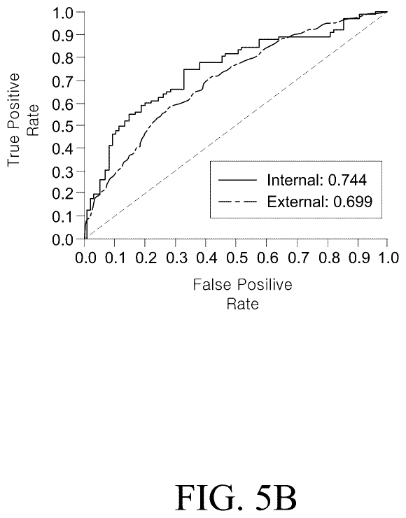

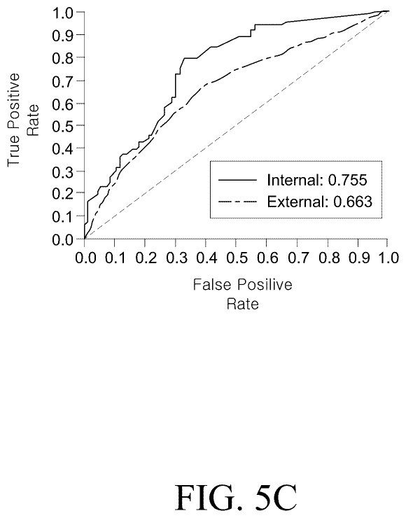

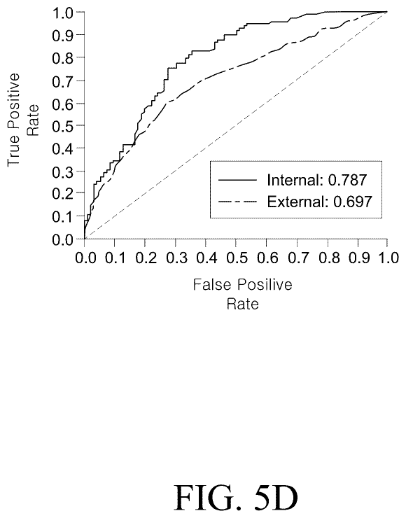

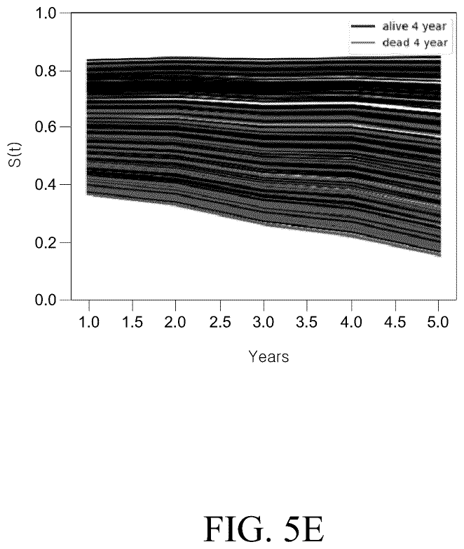

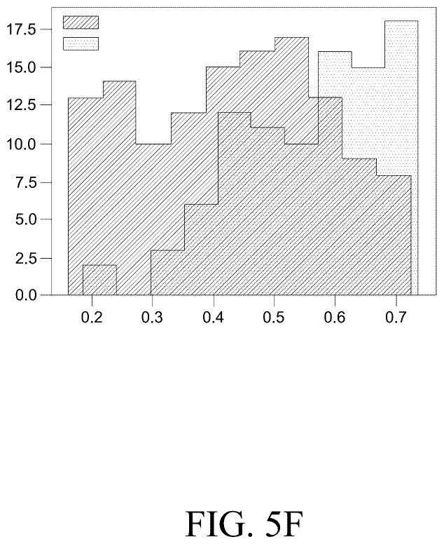

Hereinafter, with reference to FIGS. 5A to 5F, the evaluation results of the prediction model applied to various embodiments of the present disclosure will be described.

FIGS. 5A to 5F illustrate evaluation results for the prediction model applied to the

information provision method according to various embodiments.

First, the performance evaluation results for the prediction of heart failure prognosis of 4-year survival rate based on conventional echocardiographic parameters (EP) are illustrated in FIG. 5A, and the performance evaluation results for the combination of conventional EP and BRF (Baseline Risk Factors)-based prediction of age and sex are illustrated in FIG. 5B.

Referring to FIG. 5A, the EP-based model illustrates a prediction performance of AUC 0.608 in the internal validation criterion and AUC 0.603 in the external validation criterion, and referring to FIG. 5B, the EP+BRF combined model also illustrates an internal AUC of 0.744 and an external AUC of 0.699.

This means that conventional marker-based prediction models defined by specialists have low generalization performance and have limitations in reliable prognosis prediction when applied to actual clinical practice. These results may suggest structural limitations for fragmentary variable-based prediction due to the insufficient use of temporal/visual information in images in conventional technologies.

Meanwhile, in contrast, referring to FIG. 5C, the performance evaluation results for predicting the prognosis of heart failure with 4-year survival rate for the cardiac ultrasound video-based prediction model are illustrated.

At this time, the prediction model applied to various embodiments of the present disclosure illustrates the performance of internal verification criterion AUC 0.755 and external verification criterion AUC 0.663 despite using only single image information as input, and is illustrated to have superior predictive power compared to existing EP and BRF-based models.

In particular, the prediction model appears to be able to reliably predict heart failure prognosis without separate biochemical variables or clinical indicators by automatically extracting visual and temporal features from cardiac ultrasound video images without human-defined clinical indicators.

Meanwhile, the model that additionally combines BRF (sex and age) information illustrates that the AUC increases to 0.787 internally and 0.697 externally, but it appears that the image-based single model alone secures superior performance compared to existing models.

That is, the above results may suggest that the prediction model used in various embodiments of the present disclosure may have clinical value with only the image data itself.

Next, referring to FIGS. 5E and 5F, evaluation results for survival probability-based curves and mortality risk score distributions generated by the prediction model of the present disclosure are illustrated.

Referring to FIG. 5E, the cumulative survival curves of the population classified by survival status are visualized, and the survival probability curves calculated by the prediction model illustrate a clear distribution difference between the actual survival group and the dead group. In other words, the predicted cumulative survival probability illustrates a pattern of being significantly classified by actual survival status.

Referring further to FIG. 5F, a histogram comparing the distribution of the mortality risk score calculated by the prediction model according to survival status is illustrated. The death group is concentrated in the high-risk score range, while the survival group is distributed in the relatively low score range.

The above results may suggest that the risk score and survival probability curve calculated by the prediction model according to various embodiments of the present disclosure effectively reflect actual prognostic information.

That is, the present disclosure may provide rapid and reliable analysis results for the prognosis of heart failure based on cardiac ultrasound video images regardless of the skill level of the medical staff. Accordingly, the present disclosure may automatically learn visual and temporal patterns of cardiac function using the prediction model without clinical variables or hand-crafted features defined by a clinician in advance, thereby providing more precise and consistent prediction results.

In addition, the present disclosure may automatically determine the survival probability, cumulative survival curve, or mortality risk score of an image unit based on the prediction results derived from the frame unit, and provide them in the form of an intuitive graphical user interface (GUI), thereby supporting medical staff to make faster and more accurate clinical decisions.

Therefore, the present disclosure may contribute to establishing a treatment strategy for patients with heart failure by overcoming the limitations of existing conventional prognosis prediction systems and providing a quantitative and automated prediction system that may be implemented using only ultrasound images.

Although the embodiments of the present disclosure have been described in more detail with reference to the attached drawings, the present disclosure is not necessarily limited to these embodiments, and various modifications may be made without departing from the technical idea of the present disclosure. Accordingly, the embodiments disclosed in the present disclosure are not intended to limit the technical idea of the present disclosure, but to explain it, and the scope of the technical idea of the present disclosure is not limited by these embodiments. Therefore, it should be understood that the embodiments described above are exemplary in all aspects and not restrictive. The protection scope of the present disclosure should be interpreted by the following claims, and all technical ideas within a scope equivalent thereto should be interpreted as being included in the scope of the rights of the present disclosure.

What is claimed is:

1. A method for providing prognostic information on heart failure implemented by a processor, the method comprising:
receiving an echocardiographic video image of an individual suffering from heart failure; and
determining data on the prognosis of heart failure based on the received echocardiographic video image, using a prediction model trained to output data on the prognosis of the heart failure by taking the echocardiographic video image as a single input without any other input.

2. The method according to claim 1, wherein the echocardiographic video image is a cardiac ultrasound video including a plurality of frames, and
the prediction model includes
a 3D encoder configured to extract features for each of the plurality of frames of a received 3D echocardiographic video image, and
a transformer configured to derive a temporal relationship for each of the plurality of frames so that an integrated time series feature is generated.

3. The method according to claim 2, wherein the prediction model further includes a spatial attention pooling configured to generate an integrated spatial feature by considering adjacent frame features for one selected frame among the plurality of frames.

4. The method according to claim 1, wherein the data on the prognosis of the heart failure includes at least one of a survival probability within a predetermined period, a cumulative survival curve for the survival probability, a hazard ratio, and a mortality risk score.

5. The method according to claim 1, wherein the data on the prognosis of the heart failure is provided in the form of a graphical user interface (GUI) in which a mortality risk at each time point is visually expressed.

6. The method according to claim 1, further comprising additionally receiving sex and age for the individual,
wherein the determining includes determining the data on the prognosis of the heart failure based on the received sex, age, and echocardiographic video image using the prediction model.

7. The method according to claim 6, wherein the determining of the data on the prognosis of the heart failure based on the received sex, age, and echocardiographic video image includes
extracting a first feature for the received sex and age using the prediction model,
extracting a second feature for the received echocardiographic video image, integrating the first feature and the second feature, and
determining the data on the prognosis of the heart failure based on the integrated feature.

8. The method according to claim 1, wherein the prediction model is a model constructed through binary classifying whether the individual survives for each of a plurality of time intervals, and training to generate a survival probability function by accumulating a binary classification result according to whether the individual survives for each time interval.

9. A device for providing prognostic information on heart failure, the device comprising:
a communication unit configured to receive an echocardiographic video image of an individual suffering from heart failure; and
a processor functionally connected to the communication unit,
wherein the processor determines data on the prognosis of heart failure based on the received echocardiographic video image using a prediction model trained to output data on the prognosis of heart failure by taking the echocardiographic video image as a single input without any other input.

10. The device according to claim 9, wherein the echocardiographic video image is a cardiac ultrasound video including a plurality of frames, and
the prediction model includes
a 3D encoder configured to extract features for each of the plurality of frames of a received 3D echocardiographic video image, and
a transformer configured to derive a temporal relationship for each of the plurality of frames so that an integrated time series feature is generated.

11. The device according to claim 10, wherein the prediction model further includes a spatial attention pooling configured to generate an integrated spatial feature by considering adjacent frame features for one selected frame among the plurality of frames.

12. The device according to claim 9, wherein the data on the prognosis of the heart failure includes at least one of a survival probability within a predetermined period, a cumulative survival curve for the survival probability, a hazard ratio, and a mortality risk score.

13. The device according to claim 9, wherein the data on the prognosis of the heart failure is provided in the form of a graphical user interface (GUI) in which a mortality risk at each time point is visually expressed.

14. The device according to claim 9, wherein the communication unit is further configured to additionally receive sex and age for the individual, and
the processor is configured to determine the data on the prognosis of the heart failure based on the received sex, age, and echocardiographic video image using the prediction model.

15. The device according to claim 14, wherein the processor is further configured to
extract a first feature for the received sex and age using the prediction model,
extract a second feature for the received echocardiographic video image,
integrates the first feature and the second feature, and
determine the data on the prognosis of the heart failure based on the integrated feature.

16. The device according to claim 9, wherein the prediction model is a model constructed to binary-classify whether the individual survives for each of a plurality of time intervals and generate a survival probability function by accumulating a binary classification result according to whether the individual survives for each time interval.
